(19) United States
(12) Patent Application Publication
(10) Pub. No.: US 2025/0049860 A1
(43) Pub. Date: Feb. 13, 2025

(54) METHOD FOR ISOLATING, MAINTAINING, PROLIFERATING, AND DIFFERENTIATING MONOCLONAL CELLS DERIVED FROM HUMAN SALIVARY GLAND EPITHELIAL STEM CELLS OR PROGENITOR CELLS AND PRODUCTION METHOD FOR EXTRACELLULAR VESICLES FOR TREATING SALIVARY GLAND DISEASES

(71) Applicant: INDUSTRY-ACADEMIC COOPERATION FOUNDATION, YONSEI UNIVERSITY, Seodaemungu, Seoul (KR)

(72) Inventors: Jae-Yol LIM, Seoul (KR); Yeo-Jun YOON, Seoul (KR); Yongpyo HONG, Seoul (KR); Ye-Jin JEONG, Seoul (KR)

(21) Appl. No.: 18/711,487

(22) PCT Filed: Nov. 18, 2022
(86) PCT No.: PCT/KR2022/018276

§ 371 Date: May 17, 2024

(30) Foreign Application Priority Data
Nov. 19, 2021 (KR) 10-2021-0160164
Oct. 17, 2022 (KR) 10-2022-0133335
Nov. 17, 2022 (KR) 10-2022-0154264

(51) Int. Cl. A61K 35/38; C12N 5/071

(52) U.S. Cl. CPC A61K 35/38; C12N 5/0633; C12N 2501/15; C12N 2501/155

(57) ABSTRACT

The present disclosure relates to a method for isolating, maintaining, proliferating and differentiating monoclonal cells derived from human salivary gland epithelial stem cells or progenitor cells, and a production method for extracellular vesicles for treating salivary gland diseases. More specifically, conditions for isolating monoclonal salivary gland epithelial cells through an optimized subfractionation culturing method from a small amount of major salivary gland or minor salivary gland tissue obtained during surgery or biopsy processes have been established, and culture medium conditions for enabling long-term culture of salivary gland epithelial stem cells or progenitor cells in 2D have been established. Such high-purity monoclonal cells derived from human salivary gland epithelial stem cells or precursor cells are expected to be utilized not only as cell therapy products for fundamental treatment of salivary gland dysfunction and as candidates for extracellular vesicles, but also widely throughout salivary gland studies such as disease modeling, pathology research, drug screening, toxicity evaluation, and genetic manipulation.

### TECHNICAL FIELD

The present disclosure relates to a method for isolating epithelial basal stem cells or progenitor cells of human major and minor salivary glands in the form of monoclonal cells, maintaining long-term culture in vitro, and proliferating the cells, and a production method for extracellular vesicles for treating salivary gland diseases using the same.

### BACKGROUND ART

A “stem cell” is an undifferentiated cell that has the capacity to differentiate into various cells and the ability to self-replicate by constantly proliferating symmetrically or asymmetrically into cells like itself. Stem cells are present in all parts of the developing fetus, and in adulthood, mesenchymal stem cells (MSCs) are present in bone marrow, adipose tissue, and the interstitium of tissues, while epithelial stem cells (ESCs) are located in the parenchyma of each tissue. Research using stem cells has been actively conducted in various fields since the concept of stem cells was established until now. In particular, research is being conducted on extracellular vesicles, which are known to play an important role in tissue regeneration by direct differentiation of stem cells, anti-inflammation and anti-fibrosis by paracrine secretion, and intercellular signal transduction and microenvironment regulation. Stem cells may be broadly categorized into embryonic stem cells, adult stem cells, and induced pluripotent stem cells. Among them, the embryonic stem cells are extracted from human embryos and have the disadvantage of limiting the expansion of research due to bioethical controversies and limitations on tumorigenicity. Induced pluripotent stem cells are stem cells that have acquired the pluripotency of embryonic stem cells by artificially manipulating specific genes of adult stem cells to induce the phenomenon of dedifferentiation, which is a return to an undifferentiated state. However, the research of induced pluripotent stem cells involves costly issues such as facilities, equipment, and researcher skill. The adult stem cells are also called tissue-specific stem cells because they have a defined range of cells being able to differentiate compared to the two types of stem cells described above. The adult stem cells are relatively free from ethical controversies and are able to be transplanted by themselves and solve immune rejection that may occur when utilizing other people's stem cells. Further, these cells have the advantage of not requiring a high level of cost or skill to conduct research.

Stem cell therapies are emerging as candidates for treating incurable, degenerative diseases for which there is currently no cure. However, there are also a number of limitations. Currently, the most widely used method for isolating adult stem cells for stem cell therapeutic agents is the density-gradient centrifugation method (GCM), in which cells that are separated using centrifugation are sorted using antibodies or fluorescence-activated cell sorting (FACS) which is a type of flow cytometry, and the cells that adhere to the bottom of an incubator are separated and cultured in large quantities. This method has obvious limitations in terms of stem cell purity, namely the inability to exclude non-stem cells. It has been noted that the limitations of existing cell therapeutics may be due to the purity of stem cells, indicating a need for improvement in this area. The subfractionation culturing method (SCM) was designed as a way to compensate for this need. SCM is a technique for selecting only stem cells by moving cells through several stages, and has the advantage of isolating stem cells with higher purity compared to GCM. This suggests that SCM could have a fundamental impact on improving the effectiveness of stem cell therapy.

Conventional SCM utilization has focused on “mesenchymal stem cells” (MSCs), which are found in bone marrow, fat, and cord blood. It secretes various signal transmitters or growth factors such as ‘cytokines’ and ‘chemokines’ into the surrounding environment, inducing regeneration or differentiation of specific tissues, as well as immune tolerance or immunosuppression, and thus various utilization methods are being sought.

The main cells responsible for the function of the salivary glands are epithelial cells, which are divided into cells with different functions and characteristics, including acinar epithelial cells, ductal epithelial cells, myoepithelial cells, which squeeze the acinar epithelial cells and secrete saliva, and neuronal cells, which secrete neurotransmitters to the myoepithelial cells and acinar epithelial cells. Salivary gland hypofunction is a condition caused by the damage and depletion of epithelial cells, especially acinar epithelial cells, which play a major role in salivary secretion, and epithelial stem cells (ESCs), which play a major role in repairing damage to the salivary glands, due to a number of factors, including medications, radiation therapy, aging, and autoimmune diseases. This leads to decline in quality of life and frequent oral diseases due to decreased saliva secretion ability and reduced resilience. To this end, temporary symptomatic relief such as artificial saliva is being attempted, but a fundamental cure is not yet available. Stem cell therapy is emerging as a fundamental treatment for this, which is known that mesenchymal stem cells can secrete tissue growth factors or cytokines to mitigate damage or regenerate epithelial cells in tissues. A growing number of studies have shown that acinar epithelial cells or ductal epithelial cells have the characteristics of progenitor cell and stem cell and play a role in tissue regeneration. However, the direct development of these epithelial progenitor cells or stem cells, or stem cell derivatives such as exosomes, into therapeutic agents remains challenging.

A major reason why epithelial stem cells have been difficult to develop into therapeutic agents is the difficulty of isolating tissue-specific epithelial stem cells from small amounts of tissue and culturing monoclonal epithelial stem cells. Above all, culturing epithelial stem cells in two-dimensional format for a long-term is very challenging technology. Therefore, the technology to isolate small amounts of salivary gland tissue-derived epithelial stem cells from patients' biopsies into high-purity monoclonal cells, maintain and proliferate the isolated cells in two-dimensional culture conditions would be useful. In addition, when epithelial stem cells having excellent proliferative capacity, excellent extracellular vesicle secretion, tissue-specificity, and differentiation capacity into cells in tissues are capable of being isolated, proliferated, and utilized as cell therapeutic agents or extracellular vesicle therapeutic agents, it is expected to be utilized as one of the fundamental treatments for salivary gland dysfunction, as well as for embryology using stem cells in general salivary gland-related research. To achieve this goal, the main challenges include isolating and culturing monoclonal cells of epithelial stem cells from small amounts of salivary gland tissue, maintaining proliferative capacity for long-term in vitro culture and mass production of extracellular vesicles, and ensuring that the cultures do not contain heterogeneous substances for clinical application.

### DISCLOSURE

### Technical Problem

An object of the present disclosure is to specifically isolate high purity human salivary gland epithelial stem or progenitor cell-derived monoclonal cells from small amounts of salivary gland tissue by optimizing the subfractionation culturing method, followed by culture using chemically defined culture media and a combination of small molecule compounds, thereby minimizing the content of foreign substances (bovine serum, bovine pituitary extract, and porcine derived materials), and ultimately, to provide a method for long-term culture, proliferation, reproduction of differentiation capacity while maintaining the properties of salivary gland epithelial stem cells in an animal free environment, and obtaining extracellular vesicles in large quantities.

### Technical Solution

To achieve the above object, the present disclosure provides a culture medium composition for isolating, maintaining, proliferating or differentiating monoclonal cells derived from salivary gland epithelial stem cells or progenitor cells, comprising, as an active ingredient: a medium containing Y-27632, A83-01 and a bone morphogenetic protein (BMP) inhibitor.

Further, the present disclosure provides a method for isolating, maintaining, and proliferating monoclonal cells derived from salivary gland epithelial stem cells or progenitor cells, comprising: (1) obtaining salivary gland epithelial stem cells or progenitor cells from salivary gland tissue; (2) isolating salivary gland epithelial monoclonal cells by culturing the obtained salivary gland epithelial stem cells or progenitor cells in the culture medium composition as described above; and (3) maintaining or proliferating the salivary gland epithelial cells by culturing the isolated salivary gland epithelial monoclonal cells in the culture medium composition as described above.

Further, the present disclosure provides a production method for extracellular vesicles from monoclonal cells derived from salivary gland epithelial stem cells or progenitor cells, comprising: (1) obtaining salivary gland epithelial stem cells or progenitor cells from salivary gland tissue; (2) isolating salivary gland epithelial monoclonal cells by culturing the obtained salivary gland epithelial stem cells or progenitor cells in the culture medium composition as described above; (3) maintaining or proliferating the salivary gland epithelial cells by culturing the isolated salivary gland epithelial monoclonal cells in the culture medium composition as described above; and (4) isolating extracellular vesicles from the maintained or proliferated salivary gland epithelial cells described above.

Further, the present disclosure provides a pharmaceutical composition for preventing or treating salivary gland inflammatory diseases, comprising, as an active ingredient: culture medium of monoclonal cells derived from salivary gland epithelial stem cells or progenitor cells isolated, maintained, and proliferated according to the method as described above; or extracellular vesicles produced according to the method as described above.

### Advantageous Effects

The present disclosure relates to a method for isolating, maintaining, proliferating and differentiating monoclonal cells derived from human salivary gland epithelial stem cells or progenitor cells, and a production method for extracellular vesicles for treating salivary gland diseases. More specifically, conditions for isolating monoclonal salivary gland epithelial cells through an optimized subfractionation culturing method from a small amount of major salivary gland or minor salivary gland tissue obtained during surgery or biopsy processes have been established, and culture medium conditions for enabling long-term culture of salivary gland epithelial stem cells or progenitor cells in 2D have been established. The cultured stem cells were confirmed to be monoclonal salivary gland basal stem or progenitor cells, and were confirmed to have excellent yield and cell proliferative capacity. The characterization analysis validated the isolation method of epithelial stem cells and a method of differentiating cells into various cells in a three-dimensional culture environment was presented. Finally, the process of obtaining extracellular vesicles and healing effects thereof are described. Such high-purity monoclonal cells derived from human salivary gland epithelial stem cells or precursor cells are expected to be utilized not only as cell therapy products for fundamental treatment of salivary gland dysfunction and as candidates for extracellular vesicles, but also widely throughout salivary gland studies such as disease modeling, pathology research, drug screening, toxicity evaluation, and genetic manipulation.

### DESCRIPTION OF DRAWINGS

FIG. 1 is a schematic diagram of obtaining monoclonal cells of salivary gland epithelial cells through the subfractionation culturing method (SCM).

FIG. 2 shows the growth limit of salivary gland epithelial cells in each chemically defined medium (CDM) without a combination of small molecule compounds.

FIG. 3 shows the results of concentration screening of small molecule compounds using Dermacult.

FIG. 4 shows the results of concentration screening under small molecule compound combination conditions.

FIG. 5 shows the growth limit of salivary gland epithelial cells increased by the combination of small molecule compounds in each CDM.

FIG. 6 shows the results of the population doubling time shortened by the small molecule compound combination conditions.

FIG. 7 shows the results of concentration screening under small molecule compound combination conditions.

FIG. 8 shows the results of promoting cell growth by the addition of WNT during cell separation with initial SCM.

FIG. 9 shows the results of characterization of cultured cells through flow cytometry.

FIG. 10 shows the results of identifying basal cell gene expression markers by parotid gland single-cell transcriptome analysis.

FIG. 11 shows the results of identifying the salivary gland basal(progenitor) stem cells by immunofluorescence staining.

FIG. 12 shows the results of verification of salivary gland epithelial stem cell markers by flow cytometry.

FIG. 13 shows the gene expression differential results by Bulk-RNA sequencing.

FIG. 14 shows the results of characterization analysis of cells different for each colony.

FIG. 15 shows the results of verification of salivary gland epithelial stem cell markers through flow cytometry.

FIG. 16 shows the comparison of exosome production between K-SFM and Dermacult and the results of increasing exosome production by small molecule compounds.

FIG. 17 shows the results of extraction and verification of exosomes from salivary gland epithelial stem cells.

FIG. 18 shows the results of anti-inflammatory effects by exosomes in a salivary gland ligation model.

### BEST MODE

The present inventors tested four kinds of chemically defined media (CDMs) as basal media capable of culturing epithelial cells, and completed the present disclosure by optimizing a culture medium composition for isolating, maintaining, proliferating, or differentiating salivary gland epithelial stem cells or progenitor cells into monoclonal cell populations, comprising, as an active ingredient: a medium containing the growth factor WNT3A, the small molecule compound Y-27632 or similar ROCK I/II inhibitors, A83-01 or similar TGF-beta pathway inhibitors, and BMP pathway inhibitors such as DMH1 and LDN193189.

The present disclosure provides a culture medium composition for isolating, maintaining, proliferating, or differentiating monoclonal cells derived from salivary gland epithelial stem cells or progenitor cells, comprising, as an active ingredient: a medium containing Y-27632, A83-01 and a bone morphogenetic protein (BMP) inhibitor.

Preferably, the culture medium composition may comprise, but is not limited to, Y-27632 at a final concentration of 1 to 20 μM and A83-01 at a final concentration of 0.2 to 2 μM.

Preferably, the BMP inhibitor may be, but is not limited to, DMH1 or LDN19318. More preferably, the culture medium composition may comprise, but is not limited to, DMH1 at a final concentration of 0.1 to 2 μM or LDN193189 at a final concentration of 0.05 to 0.5 μM.

Preferably, the medium may be applied to a subfractionation culturing method (SCM) but is not limited thereto.

Further, the present disclosure provides a method for isolating, maintaining, and proliferating monoclonal cells derived from salivary gland epithelial stem cells or progenitor cells, comprising: (1) obtaining salivary gland epithelial stem cells or progenitor cells from salivary gland tissue; (2) isolating salivary gland epithelial monoclonal cells by culturing the obtained salivary gland epithelial stem cells or progenitor cells in the culture medium composition as described above; and (3) maintaining or proliferating the salivary gland epithelial cells by culturing the isolated salivary gland epithelial monoclonal cells in the culture medium composition as described above.

Preferably, in step (2), WNT3A may be added to culture the salivary gland epithelial stem cells or progenitor cells, but the present disclosure is not limited thereto.

Preferably, the culturing of the salivary gland epithelial stem cells or progenitor cells in step (2) may be performed according to the subfractionation culturing method (SCM), but the present disclosure is not limited thereto.

Preferably, the monoclonal cells derived from salivary gland epithelial stem cells or progenitor cells may be CD49f+/CD26− salivary gland basal cells, but the present disclosure is not limited thereto.

Preferably, the monoclonal cells derived from salivary gland epithelial stem cells or progenitor cells may express salivary gland basal cell markers and salivary gland progenitor cell markers KRT14, KRT5, KRT19, SOX9, and TP63, and may express a salivary gland epithelial cell marker CDH1, but the present disclosure is not limited thereto.

Preferably, the monoclonal cells derived from salivary gland epithelial stem cells or progenitor cells may have multipotency into salivary gland epithelial tissue.

Further, the present disclosure provides a production method for extracellular vesicles from monoclonal cells derived from salivary gland epithelial stem cells or progenitor cells, comprising: (1) obtaining salivary gland epithelial stem cells or progenitor cells from salivary gland tissue; (2) isolating salivary gland epithelial monoclonal cells by culturing the obtained salivary gland epithelial stem cells or progenitor cells in the culture medium composition as described above; (3) maintaining or proliferating the salivary gland epithelial cells by culturing the isolated salivary gland epithelial monoclonal cells in the culture medium composition as described above; and (4) isolating extracellular vesicles from the maintained or proliferated salivary gland epithelial cells described above.

Preferably, the extracellular vesicle may be a salivary gland epithelial stem cell exosome, but are not limited thereto.

The term “extracellular vesicle (EV)” in the present disclosure refers to a nano-sized vesicle derived from a cell, and may be classified into exosomes, microvesicles, ectosomes, microparticles, membrane vesicles, nanovesicles, outer membrane vesicles, and the like, depending on secretion form and size thereof. The extracellular vesicle serves as a primary mode of communication between cells, including nucleic acids and proteins, which are the main components of cells.

Further, the present disclosure provides a pharmaceutical composition for preventing or treating salivary gland inflammatory diseases, comprising, as an active ingredient: culture medium of monoclonal cells derived from salivary gland epithelial stem cells or progenitor cells isolated, maintained, and proliferated according to the method as described above; or extracellular vesicles produced according to the method as described above.

The pharmaceutical compositions of the present disclosure may further comprise a pharmaceutically acceptable carrier, and the pharmaceutically acceptable carrier is any one conventionally utilized in formulation, such as lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methylhydroxybenzoate, propylhydroxybenzoate, talc, magnesium stearate, and mineral oils, but is not limited thereto. The composition for preventing or treating cancer metastasis of the present disclosure may further comprise lubricants, wetting agents, sweeteners, flavoring agents, emulsifiers, suspending agents, preservatives, and the like, in addition to the components above.

The pharmaceutical composition of the present disclosure may be administered orally or parenterally, and if parenterally, may be administered by intravenous infusion, subcutaneous injection, intramuscular injection, intraperitoneal injection, endothelial administration, topical administration, direct intratubular injection, intranasal administration, intrapulmonary administration, and intrarectal administration. When administered orally, proteins or peptides are digestible, and thus oral compositions may be formulated to coat the active agent or to protect it from degradation in the stomach, and the composition of the present disclosure may be administered by any device that allows the active substance to be transported to the target cells.

Suitable dosages of the pharmaceutical compositions of the present disclosure vary depending on factors such as method of formulation, mode of administration, patient's age, weight, sex, pathological condition, food, administration time, administration route, excretion rate, and reaction sensitivity, and the ordinarily skilled physician may readily determine and prescribe a dosage effective for the desired treatment or prevention.

The pharmaceutical compositions of the present disclosure may be formulated with pharmaceutically acceptable carriers and/or excipients to be prepared in unit dose form or prepared by introducing them into multi-dose containers, in accordance with methods readily practiced by a person skilled in the art to which the present disclosure pertains. In this case, the formulation may be in the form of a solution, suspension, or emulsion in an oil or aqueous medium, or in the form of an extract, acid, suspension, powder, granule, tablet, or capsule, and may further comprise a dispersant or a stabilizing agent.

The “saliva” of the present disclosure is a mixed fluid secreted by the parotid gland, submaxillary gland, sublingual gland, and mucous gland present in the oral mucosa. Saliva is a key component of the human body and is produced in the salivary glands and discharged into the oral cavity. Saliva is an essential component of the human body and contains bioactive proteins, digestive enzymes, mucus, immunoglobulins, and various salts.

Saliva plays a very important role not only in oral health but also in maintaining homeostasis in the human body. For example, saliva's main components, mucin and immunoglobulins, act as a primary defense against external infections and protect the oral mucosa and teeth by lubricating the mouth and teeth, maintaining moisture, and neutralizing pH. In addition, saliva contains digestive enzymes such as amylase, such as ptyalin, which facilitates digestion by breaking down starch into maltose units. In addition, the secretion of saliva may regulate the body's water metabolism and body temperature and excrete toxic substances (I, Hg, Pb, etc.).

The “salivary gland” of the present disclosure is an organ that produces and secretes saliva and is classified as a major salivary gland such as parotid gland, submaxillary gland, and sublingual gland, and minor salivary glands, which are distributed in different parts of the oral mucosa, such as the mucous glands, which are located in the mucous membrane of the oral cavity.

### MODE FOR CARRYING OUT THE INVENTION

Hereinafter, the present disclosure will be described in detail with reference to embodiments for better understanding. However, the following exemplary embodiments are only illustrative of the present disclosure and the scope of the present disclosure is not limited to the following embodiments. Examples of the present disclosure are provided to more completely explain the present disclosure to those skilled in the art.

### EXPERIMENTAL EXAMPLES

The following Experimental Examples are intended to provide Experimental Examples commonly applied to each of Examples according to the present disclosure.

### 1. Reagent

The following reagents were used in the present disclosure.

Keratinocyte SFM (1×) without calcium chloride (#10725-018, Gibco), Epidermal growth factor (EGF) (#10450-013, Gibco), Bovine pituitary extract (BPE) (#13028-014, Gibco), Recombinant Human Wnt-3a Protein (#5036-WN-010, R&D), EpiLife™ Medium with 60 uM Calcium (#MEPI500CA, Gibco), EpiLife™ Defined Growth Supplement Medium (#S0125, Gibco), DermaCult™ Keratinocyte Expansion Medium (#100-0500, STEMCELL), DermaCult™ Keratinocyte Expansion Supplement (#100-0502, STEMCELL), CnT-Prime Epithelial Proliferation Medium (#CnT-PR, CellnTec), Primocin (#anti-pm, Invivogen), Y-27632 dihydrochloride (#TB1254-GMP, Tocris), A83-01 (#TB2939-RMU, Tocris), DMH-1 (#4126, Tocris), LDN 193189 dihydrochloride (#TB6053-GMP, Tocris), CHIR i99021 (#4423, Tocris), PD0325901 (#4192, Tocris), Isoproterenol hydrochloride (#1741, Tocris), CTS™ DPBS, calcium, magnesium (#A1285801, Gibco), CTS™ DPBS (1×), without calcium chloride, without magnesium chloride (#A1285601, Gibco), Collagenase NB6 GMP grade (N0002779, Nordmark), CTS™ TrypLE™ Select Enzyme (#A1285901, Gibco), 100×20 mm culture dish (#150466, Thermo), 48 well culture plate (#30048, SPL), 24 well culture plate (#30024, SPL), 12 well culture plate (#30012, SPL), 6 well culture plate (#30006, SPL), 25 cm² cell culture plate (#70025, SPL), 75 cm² cell culture plate (#70075, SPL), 175 cm² cell culture plate (#70175, SPL), STEM-CELLBANKER-GMP Grade (#11924, AMSBIO), Scalpel blade (feather #FM.4), Strainer (#93070, SPL), Falcon® 5 mL Round Bottom Polystyrene Test Tube, with Cell Strainer Snap Cap (#352235, Falcon), Protein LoBind Tube 1.5 mL (#022431081, Eppendorf AG), 5 mL conical Tube (#51105, SPL), 15 mL conical Tube (#51115, SPL), and 50 mL conical Tube (#51150, SPL).

2. Obtaining Human Salivary Gland Epithelial Cells from Tissue
(1) Normal tissue from the main salivary gland that was not invaded by tumor was collected by biopsy during surgical procedures such as tumor removal, or 1-2 lobes were collected from the minor salivary gland of a patient with suspected Sjogren's, and then the collected tissues were put in CTS™ DPBS, calcium, and magnesium and stored in a refrigerator at 4° C. until the experiment was performed on the same day or the next day.
(2) Collagenase was added to CTS™ DPBS, calcium, and magnesium to prepare 0.4 U/mL of enzyme solution. Y27632 was added to the corresponding enzyme solution to achieve a concentration of 10 μM. 1 mL of solution was prepared for every 10 mg of tissue, provided that a minimum volume of 1 mL was prepared if the tissue weighed less than 10 mg.
(3) The tissue was placed in a petri dish, minced using a scalpel blade, and added to the prepared enzyme solution and reacted at 37° C. for 1 hour. The resulting product was inverted every 10 minutes to mix the solution and tissue well.
(4) The mixed product was centrifuged at 300 g for 5 minutes at 4° C. to remove the enzyme solution, and centrifuged again at 300 g for 5 minutes at 4° C. after adding CTS™ DPBS (1×), without calcium chloride, without magnesium chloride, in twice the volume of the enzyme solution, and removed the DPBS.
(5) CTS™ TrypLE™ Select Enzyme was added in an amount calculated as 1 mL per 50 mg of original tissue weight. Y27632 was added to achieve a concentration of 10 μM (1 mL was added if the tissue weighed 50 mg or less). The resulting product was reacted at 37° C. for 20 minutes and inverted every 10 minutes to ensure thorough mixing.
(6) The resulting product was mixed with an equal amount of culture medium, passed through a 70 m strainer, and successively through a Falcon tube with strainer, followed by centrifugation at 300 g for 5 minutes at 4° C., and the supernatant was removed. Then, the culture medium was added once more, followed by centrifugation at 300 g for 5 minutes at 4° C. to remove the supernatant, and the cells were counted.

### 3. Isolating Human Salivary Gland Epithelial Monocytes Using SCM

(1) The cells were homogenized in 10 ml culture medium, spread evenly onto a 100×20 mm culture dish, and then incubated in an incubator at 37° C. and 5% CO2. In this case, the composition of each culture medium is as shown in Table 1.
(2) To perform the subfractionation culturing method (SCM) to isolate epithelial monoclonal cells, after 2 hours, the culture medium containing the cells was transferred to a new 100×20 mm culture dish. This procedure was repeated two more times.
(3) After 24 hours, the dish resulting from (2) was cultured, and the existing culture medium was removed every 2-3 days and replaced it with fresh culture medium.
(4) After confirming the formation of colonies, when the size of a cell cluster forming one colony exceeded 1 cm², the culture medium was removed, the cluster was marked with a hydrophobic pen, and the residual culture medium was removed using 1×PBS. 50 to 100 μL of TrypLE+Y27632 was added, and the cells were stored in an incubator at 37° C. and 5% CO₂ environment for 15 minutes.
(5) Each cell population detached from the dish by TrypLE was added to a separate 1.5 ml tube, and 1 ml of culture medium was added.
(6) After centrifugation at 300 g for 5 minutes at 4° C., the supernatant was removed, and the cells were homogenized and spread in 250 μL of fresh culture medium into each well of a 48-well plate for each cell population.

TABLE 1

| Culture | Final | | Culture | Final | Culture | Final | Culture | Final |
|---|---|---|---|---|---|---|---|---|
| medium | concentration | | medium | concentration | medium | concentration | medium | concentration |
| 1 | Basal | | 2 | Basal | 3 | Basal | 4 | Basal |
| K-SFM | medium | | EpiLife | medium | Dermacult | medium | CnT-PR | medium |
| BPE | 50 | μg/m [symbol] | EDGS | 1× | Dermacult | 1× | Primocin | 1× |
| | | | (100×) | | Supplement | | (500×) | |
| | | | | | (50×) | | | |
| EGF | 5 | ng/m [symbol] | Primocin | 1× | Primocin | 1× | Y-27632 | 10 μM |
| | | | (500×) | | (500×) | | | |
| CaCl2 | 60 | μM | Y-27632 | 10 μM | Y-27632 | 10 μM | A83-01 | 1 μM |
| Primocin | 1× | | A83-01 | 1 μM | A83-01 | 1 μM | LDN193189 | 0.1 μM |
| (500×) | | | | | | | | |
| Y-27632 | 10 | μM | LDN193189 | 0.1 μM | LDN193189 | 0.1 μM | | |
| A83-01 | 1 | μM | | | | | | |
| DMH-1 | 1 | μM | | | | | | |
| WNT3A | 100 | ng/m [symbol] | | | | | | |

### 4. Proliferating Salivary Gland Epithelial Cells Isolated as Single Cell Populations

(1) The culture medium was removed every 2-3 days and replaced it with fresh culture medium.
(2) When the wells were 80 to 90% full of cells, the cells were subcultured.
(3) In this case, the composition of each culture medium used for subculture was shown in Table 2.
(4) After removing the culture medium, the residual culture medium was removed using PBS. Then, TrypLE express (approximately 250 l) enough to be laid on the bottom of the well was added and then the cells were stored in an incubator at 37° C. and 5% CO2 for 15 minutes.
(5) The cells detached from the culture plate by TrypLE express were added to separate 5 ml tubes, respectively, and neutralized by adding 1 ml of the culture medium in Table 2.
(6) After centrifugation at 300 g for 5 minutes at 4° C., the supernatant was removed, and the cells were homogenized and spread in 1 ml of culture medium shown in Table 2 into each well of a 12-well plate for each cell population.
(7) The existing culture medium was removed every 2-3 days and replaced it with fresh culture medium.
(8) The above-described subculture was performed through a scale-up step as follows: 24 well culture plate (0.5 mL), 12 well culture plate (1 mL), 6 well culture plate (2 ml), 25 cm² cell culture flask (5 ml), 75 cm² cell culture flask (15 ml), and 175 cm² cell culture flask (35 ml). The volume of culture medium added is given in parentheses.
(9) The cells from 6 well culture plate could be used for various experiments, and the subculture was performed at a ratio of 1:2 to 1:10.
(10) When the proliferation rate did not follow an exponential pattern, as determined by population doubling assessment, the cells were judged to be senescent and discarded.

TABLE 2

| Culture | Final | | Culture | Final | Culture | Final | Culture | Final |
|---|---|---|---|---|---|---|---|---|
| medium | concentration | | medium | concentration | medium | concentration | medium | concentration |
| 1 | Basal | | 2 | Basal | 3 | Basal | 4 | Basal |
| K-SFM | medium | | EpiLife | medium | Dermacult | medium | CnT-PR | medium |
| BPE | 50 | μg/m [symbol] | EDGS | 1× | Dermacult | 1× | Y-27632 | 10 μM |
| | | | (100×) | | Supplement | | | |
| | | | | | (50×) | | | |
| EGF | 5 | ng/m [symbol] | Primocin | 1× | Primocin | 1× | A83-01 | 1 μM |
| | | | (500×) | | (500×) | | | |
| CaCl2 | 60 | μM | Y-27632 | 10 μM | Y-27632 | 10 μM | LDN193189 | 0.1 μM |
| Primocin | 1× | | A83-01 | 1 μM | A83-01 | 1 μM | | |
| (500×) | | | | | | | | |
| Y-27632 | 10 | μM | LDN193189 | 0.1 μM | LDN193189 | 0.1 μM | | |
| A83-01 | 1 | μM | | | | | | |
| DMH-1 | 1 | μM | | | | | | |
| WNT3A | 100 | ng/m [symbol] | | | | | | |

### 5. Characterization of Proliferated Epithelial Cells

(1) The following experiments were initiated when successfully proliferated clones and culture media at 70-80% confluency.
(2) The characteristics of isolated and cultured monoclonal cells derived from human salivary gland epithelial stem cells or progenitor cells were analyzed by immunofluorescence staining.
(3) RNA was isolated using TRIzol or an RNA extraction kit, and changes in gene expression were confirmed using real-time PCR and bulk-RNA sequencing.
(4) Flow cytometry was used to determine the expression of stem cell-associated proteins on the cell surface.
(5) Differentiation ability was tested by three-dimensional culture using Matrigel.
(6) As extracellular vesicles reflect the characteristics of the cell that secreted the extracellular vesicles, the extracellular vesicles were harvested and confirmed by Western blot to determine whether CD9, etc., were highly expressed.

### <Example 1> Isolation of Monoclonal Cells Derived from Human Salivary Gland Epithelial Stem Cells or Progenitor Cells and Screening of Optimal Culture Medium Composition for Prolonged In Vitro Culture

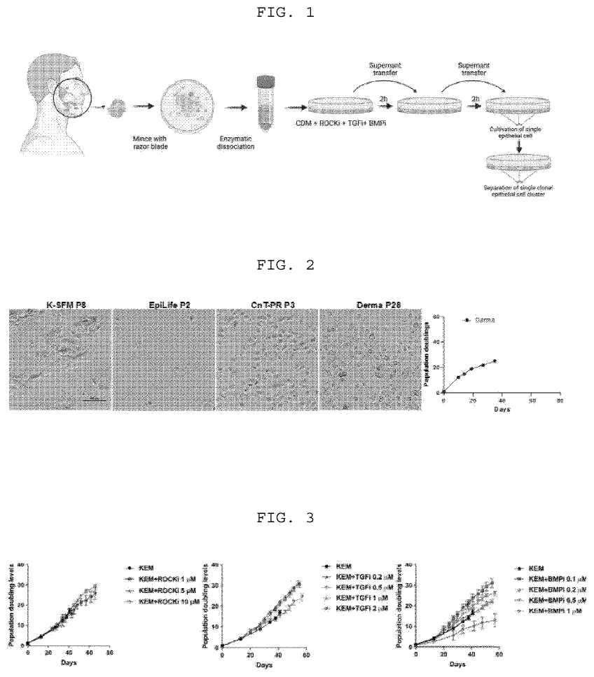

High purity monoclonal cells based on human salivary gland epithelial stem cells or progenitor cells were isolated using modified SCM and mixing method of various types of chemically defined media (CDM) and small molecule compounds (FIG. 1). During the SCM process, the culture media in [Table 1] were used for the experiments, and after the first subculture, the culture media in [Table 2] were used.

First, the extent to which the culture was possible using only CDM capable of culturing epithelial cells was evaluated. When subculture was performed in K-SFM, EpiLife, and CnT-PR at a ratio of 1:2 in the absence of small molecule compounds, it was confirmed that cells stopped growing and became senescent after 6, 2, and 3 passages on average, respectively, but for Dermacult, 20 to 30 passages were possible even without small molecule compounds (FIG. 2).

Dermacult, which can stably culture human salivary gland epithelial cells in CDM, was used as the epithelial cell culture medium (keratinocyte expansion medium, KEM), and the small molecule compounds ROCK I/II inhibitor (Y-27632), TGF-beta pathway inhibitor (A83-01), and BMP pathway inhibitor (LDN193189) were added to determine the optimal concentrations at which cell characteristics were maintained and stable long-term culture was achieved, respectively (FIG. 3).

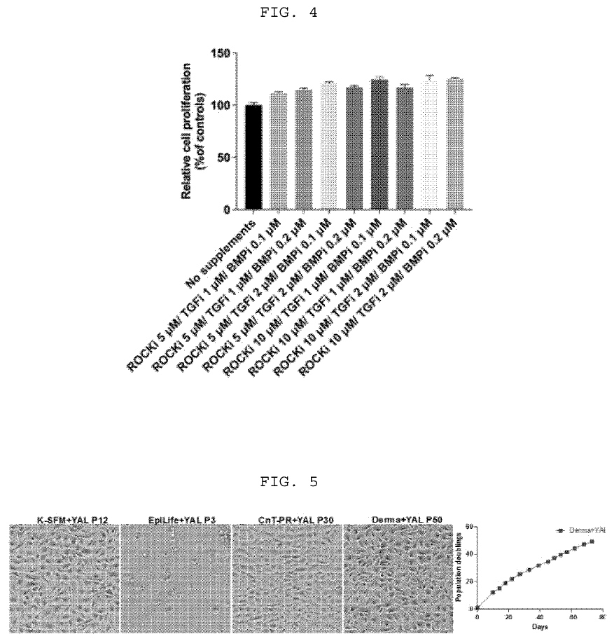

Finally, the combination experiments with different concentrations of the small molecule compounds confirmed that salivary gland epithelial cell growth was best maintained when Y-27632 10 μM, A83-01 1 μM, and LDN193189 0.1 μM were added to KEM (FIG. 4).

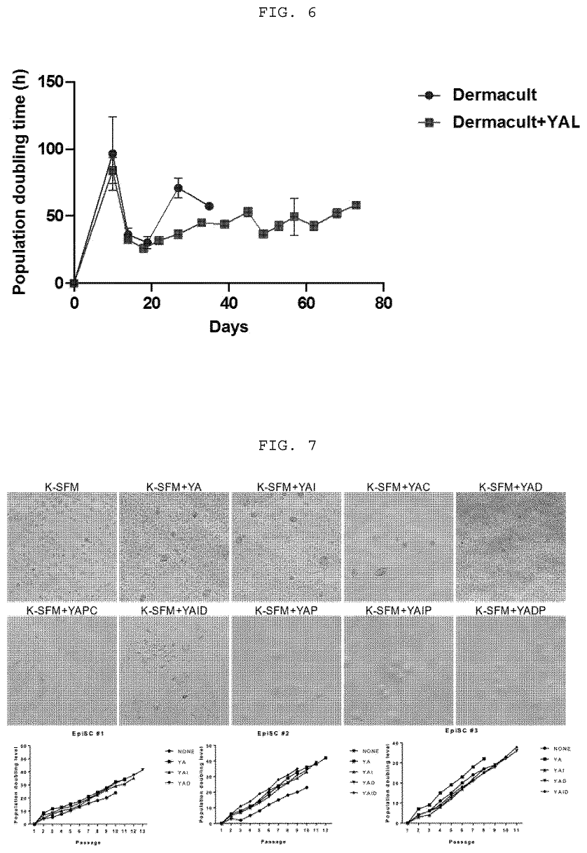

With the optimal combination of small molecule compounds, K-SFM exhibited the capability to perform between 9 and 18 subcultures, EpiLife performed 3 subcultures, CnT-PR performed over 30 subcultures, and Dermacult performed over 50 subcultures (FIG. 5).

In particular, when adding small molecule compounds, the experimental group with Dermacult confirmed that cells proliferated more rapidly and could be cultured for longer periods of time, and the population doubling time of the cells was dramatically reduced, as compared to the control cell cultured without the small molecule compounds (FIG. 6).

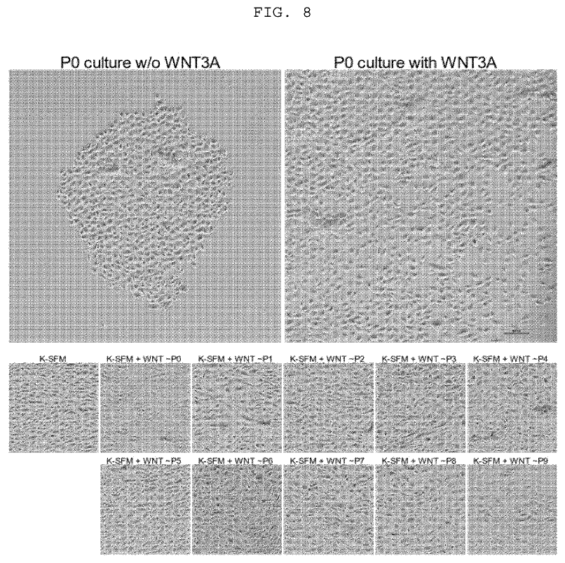

As to K-SFM among CDM, when various combinations of small molecule compound were checked, the addition of Y-27632 and A83-01 showed universally good results, whereas the addition of Chir99021 or PD0325901 showed poor results. Isoproterenol had no effect. Furthermore, when LDN193189 was replaced with DMH-1, another class of BMP pathway inhibitor, it had similar effects to Dermacult's YAL, but at a higher concentration of 1 μM (FIG. 7).

When using K-SFM among CDM, the initial addition of WNT showed rapid cell growth, but it was not essential in the culture conditions of small molecule compounds, but it was confirmed that prolonged addition of WNT rather changed the morphology of the cells. When WNT3A is to be processed, it is preferable to use it only in the SCM separation process and exclude it from the subsequent subculture process (FIG. 8).

### <Example 2> Lineage Analysis of Monoclonal Cells Derived from Human Salivary Gland Epithelial Stem Cells or Progenitor Cells by Flow Cytometry and Immunofluorescence Staining

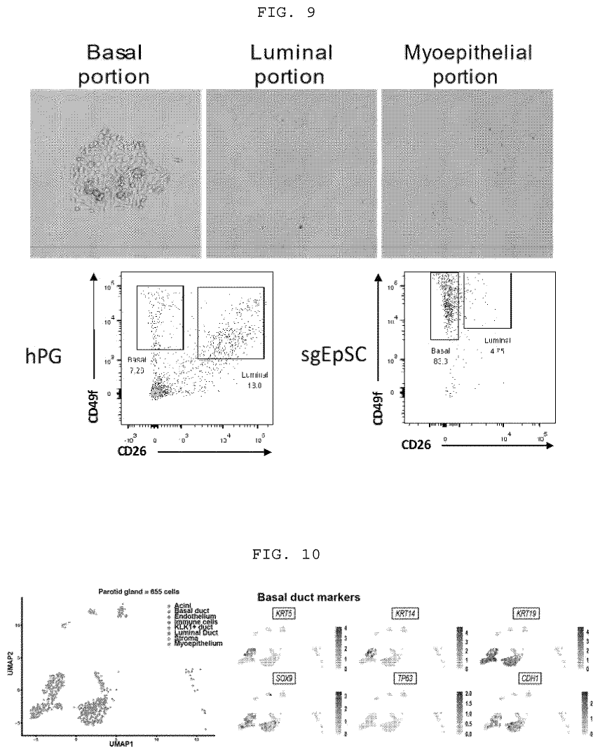

To determine whether the isolated cells were basal, luminal, myoepithelial, or acinus cells, which constitute the epithelial cells of the salivary glands, cells of parotid tissue were sorted by flow cytometry using CD49f and CD26 antibodies and cultured, and the cultured cells were further analyzed by flow cytometry and found to be composed of CD49f+/CD26− basal cells (FIG. 9).

To further verify that only salivary gland basal progenitors and stem cells are able to be selectively grown under the conditions of the culture medium established above, candidate markers specifically expressed in salivary gland basal cells were shortlisted by single-cell transcriptome analysis (FIG. 10).

Basal cell expression markers were then identified by immunofluorescent staining in cultured cells. KRT14, KRT5, KRT19, SOX9, and TP63, which are salivary gland basal cell markers and progenitor cell markers, were identified, while KRT7, a marker of differentiated ductal epithelial cells, and SOX2, a precursor marker of acinus cells, were not identified. In addition, it was confirmed by the expression of CDH1 and lack of VIM that the epithelial cells did not lose their characteristics even during long-term culture (FIG. 11).

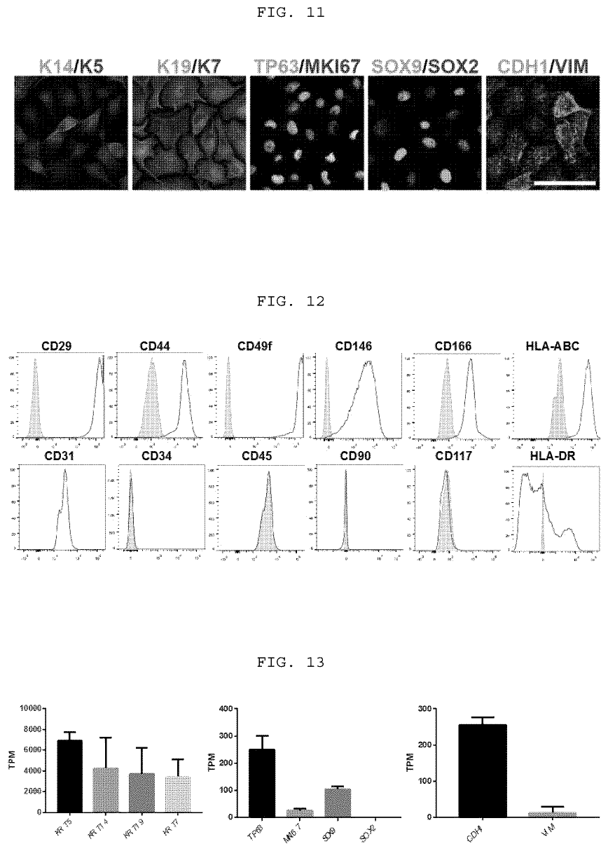

Next, to verify epithelial stem cells, flow cytometric analysis was performed and compared to mesenchymal stem cell markers, and CD34 and CD90, which are known to be positive in mesenchymal stem cells, were found to be negative (FIG. 12).

### <Example 3> Analysis of Characteristics of Monoclonal Cells Derived from Human Salivary Gland Epithelial Stem Cells or Progenitor Cells by Bulk-RNA Sequencing

The characteristics of these cells were also expressed in the same way in bulk RNA-sequencing that analyzed each of five colonies, confirming that SOX2 and VIM were not expressed, KRT14, KRT5, KRT19, SOX9, TP63, and CDH1 were highly expressed, and KRT7 was relatively low expressed (FIG. 13).

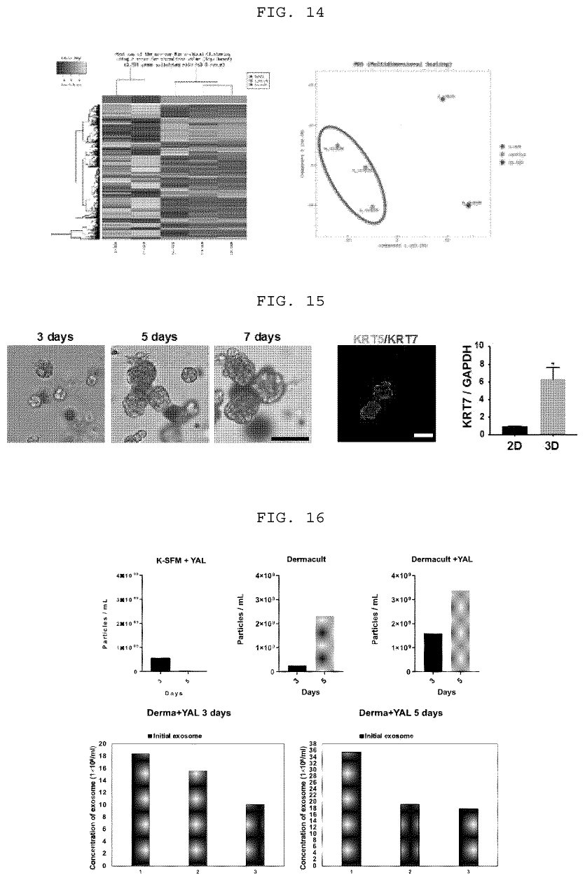

Furthermore, each colony had slightly different characteristics, reaffirming the ability to isolate monoclonal epithelial stem cells using the SCM (FIG. 14).

### <Example 4> Analysis of Differentiation Ability of Monoclonal Cells Derived from Human Salivary Gland Epithelial Stem Cells or Progenitor Cells by Immunofluorescence Staining

When organoids were produced by embedding the cells in Matrigel, it was confirmed whether luminal cells were formed from cells composed mainly of basal cells. After embedding, the growth in size of the organoids could be seen in brightfield, and at the same time, it was confirmed that KRT7 was expressed and increased through immunofluorescence staining and real-time PCR (FIG. 15).

### <Example 5> Isolation and Characterization Analysis of Extracellular Vesicles and Confirmation of Efficacy

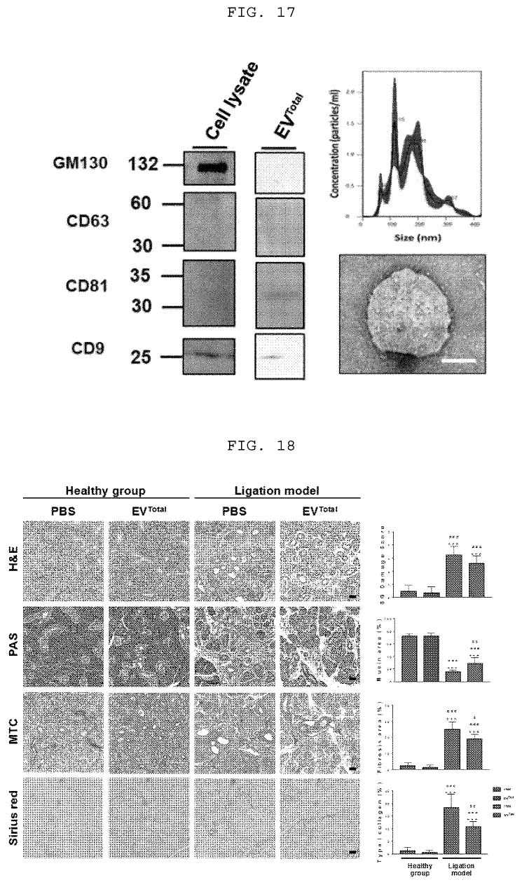

Comparing the production of exosomes in K-SFM and Dermacult, it could be confirmed that the addition of the small molecule compound combination to K-SFM produced fewer exosomes while the addition of the small molecule compound combination to Dermacult produced more exosomes, as compared to Dermacult (FIG. 16).

Extracellular vesicles produced from epithelial stem cells were confirmed to have the characteristics of epithelial stem cell exosomes by Western blot, NTA and transmission scanning microscopy (FIG. 17).

After ligation of the rat submaxillary conduit for 2 weeks, the released model showed significant anti-inflammatory, anti-fibrotic, and tissue protective effects compared to the PBS-injected control (FIG. 18).

The present disclosure enables the culture of high purity human salivary gland epithelial stem cells by a combination of small molecule compounds under serum-free and BPE-free conditions (Dermacult). It also shows potential to be successful even in conditions where animal free is achieved (CnT-PR). Further, it was found that the composition of the present disclosure could be used as a treatment for salivary gland inflammatory diseases by increasing the amount of exocytic vesicle secretion under the corresponding conditions.

While the foregoing has described in detail certain aspects of the present disclosure, it will be apparent to one of ordinary skill in the art that these specific descriptions are merely preferred embodiments and are not intended to limit the scope of the present disclosure. Therefore, the substantive scope of the present disclosure will be defined by the appended claims and equivalents thereof.

1.-14. (canceled)

15. A method for culturing monoclonal cells derived from salivary gland epithelial stem cells or progenitor cells, comprising:
(1) obtaining salivary gland epithelial stem cells or progenitor cells from salivary gland tissue;
(2) isolating monoclonal cells derived from salivary gland epithelial stem cells or progenitor cells by culturing the obtained salivary gland epithelial stem cells or progenitor cells in the culture medium containing Y-27632, A83-01, and a bone morphogenetic protein (BMP) inhibitor; and
(3) culturing the monoclonal cells derived from salivary gland epithelial stem cells or progenitor cells in the culture medium containing Y-27632, A83-01, and a bone morphogenetic protein (BMP) inhibitor.

16. The method of claim 15, wherein the BMP inhibitor is DMH1 or LDN193189.

17. The method of claim 15, wherein the BMP inhibitor is DMH1 with a concentration of 0.1 to 2 μM.

18. The method of claim 15, wherein the BMP inhibitor is LDN193189 with a concentration of 0.05 to 0.5 μM.

19. The method of claim 15, wherein the Y-27632 is at a concentration of 1 to 20 μM.

20. The method of claim 15, wherein the A83-01 is at a concentration of 0.2 to 2 μM.

21. The method of claim 15, wherein the culturing is performed by subfractionation culturing method (SCM).

22. The method of claim 15, wherein in step (2), WNT3A is added to the culture medium.

23. The method of claim 15, wherein the monoclonal cells derived from salivary gland epithelial stem cells or progenitor cells are CD49f+/CD26− salivary gland basal cells.

24. The method of claim 15, wherein the monoclonal cells derived from salivary gland epithelial stem cells or progenitor cells express any one or more selected from KRT14, KRT5, KRT19, SOX9, TP63, and CDH1.

25. The method of claim 15, wherein the monoclonal cells derived from salivary gland epithelial stem cells or progenitor cells have multipotency into salivary gland epithelial tissue.

26. A production method for extracellular vesicles from monoclonal cells derived from salivary gland epithelial stem cells or progenitor cells, comprising:
(1) obtaining salivary gland epithelial stem cells or progenitor cells from salivary gland tissue;
(2) isolating monoclonal cells derived from salivary gland epithelial stem cells or progenitor cells by culturing the obtained salivary gland epithelial stem cells or progenitor cells in the culture medium containing Y-27632, A83-01, and a bone morphogenetic protein (BMP) inhibitor;
(3) culturing the monoclonal cells derived from salivary gland epithelial stem cells or progenitor cells in the culture medium containing Y-27632, A83-01, and a bone morphogenetic protein (BMP) inhibitor; and
(4) isolating extracellular vesicles from the monoclonal cells derived from salivary gland epithelial stem cells or progenitor cells.

27. The production method of claim 26, wherein the extracellular vesicle is a salivary gland epithelial stem cell exosome.

28. The production method of claim 26, wherein the BMP inhibitor is DMH1 or LDN193189.

29. The production method of claim 26, wherein the culturing is performed by subfractionation culturing method (SCM).

30. The production method of claim 26, wherein in step (2), WNT3A is added to the culture medium.

31. A method for treating salivary gland inflammatory diseases, comprising:
administering monoclonal cells derived from salivary gland epithelial stem cells or progenitor cells; or extracellular vesicles to a subject in need thereof.

32. The method of claim 31, wherein the monoclonal cells derived from salivary gland epithelial stem cells or progenitor cells are produced by the following steps:
(1) obtaining salivary gland epithelial stem cells or progenitor cells from salivary gland tissue;
(2) isolating monoclonal cells derived from salivary gland epithelial stem cells or progenitor cells by culturing the obtained salivary gland epithelial stem cells or progenitor cells in the culture medium containing Y-27632, A83-01, and a bone morphogenetic protein (BMP) inhibitor; and
(3) culturing the monoclonal cells derived from salivary gland epithelial stem cells or progenitor cells in the culture medium containing Y-27632, A83-01, and a bone morphogenetic protein (BMP) inhibitor.

33. The method of claim 31, wherein the monoclonal cells derived from salivary gland epithelial stem cells or progenitor cells are CD49f+/CD26− salivary gland basal cells, and express any one or more selected from KRT14, KRT5, KRT19, SOX9, TP63, and CDH1.

34. The method of claim 31, wherein the extracellular vesicles are produced by the following steps:
(1) obtaining salivary gland epithelial stem cells or progenitor cells from salivary gland tissue;
(2) isolating monoclonal cells derived from salivary gland epithelial stem cells or progenitor cells by culturing the obtained salivary gland epithelial stem cells or progenitor cells in the culture medium containing Y-27632, A83-01, and a bone morphogenetic protein (BMP) inhibitor;
(3) culturing the monoclonal cells derived from salivary gland epithelial stem cells or progenitor cells in the culture medium containing Y-27632, A83-01, and a bone morphogenetic protein (BMP) inhibitor; and
(4) isolating extracellular vesicles from the monoclonal cells derived from salivary gland epithelial stem cells or progenitor cells.
